(19) United States
(12) Patent Application Publication
(10) Pub. No.: US 2006/0020297 A1
(43) Pub. Date: Jan. 26, 2006

(54) Neurostimulation system with distributed stimulators

(76) Inventors: Martin T. Gerber, Maple Grove, MN (US); William D. Weimer, Plymouth, MN (US)

Correspondence Address: SHUMAKER & SIEFFERT, P. A., 8425 SEASONS PARKWAY, SUITE 105, ST. PAUL, MN, 55125, US

(21) Appl. No.: 10/977,336

(22) Filed: Oct. 29, 2004

Related U.S. Application Data
(60) Provisional application No. 60/589,541, filed on Jul. 20, 2004.

(51) Int. Cl. A61N 1/18

(52) U.S. Cl. 607/39; 607/41

(57) ABSTRACT

The disclosure is directed to a neurostimulation system and method that make use of remote, distributed stimulators implanted at selected positions within a patient. Each stimulator is capable of independently delivering neurostimulation energy to a patient. A master controller communicates with the stimulator by wireless telemetry, and controls and synchronizes the operation of the stimulators on a selective basis to deliver a desired mode of neurostimulation energy to the patient. The distributed stimulators function as remote “slave” stimulators that can be used to selectively stimulate various nerves. In this manner, the stimulators can be selectively activated and used in a coordinated manner to provide a multi-function stimulation generator or a multi-site stimulator.

This application claims the benefit of U.S. provisional application No. 60/589,541, filed Jul. 20, 2004, the entire content of which is incorporated herein by reference.

### FIELD OF THE INVENTION

The invention relates generally to medical devices and, more particularly, to medical devices for delivery of neurostimulation therapy.

### BACKGROUND

A variety of pelvic floor disorders such as urinary control disorders, fecal control disorders, interstitial cystitis, sexual dysfunction, and pelvic pain are influenced by the sacral nerves and other nerves. The organs involved in various bodily functions in the pelvic floor region receive much of their control via the second, third, and fourth sacral nerves, commonly referred to as S2, S3, and S4, respectively. The sacrum, in general, is a large, triangular bone situated at the lower part of the vertebral column, and at the upper and back part of the pelvic cavity. The spinal canal runs throughout the sacrum. The sacral nerves pass through the sacrum via the anterior and posterior sacral foramina. Pelvic organs are also innervated via other nerves, such as the pudendal nerve.

Electrical stimulation of the sacral nerves, pudendal nerves, and other nerves of the pelvic floor has been found to offer relief for many pelvic floor disorders. For example, neurostimulation systems have been developed with medical leads having discrete electrodes that are implanted on and near the sacral nerves. An implantable pulse generator drives the electrodes with an electrical signal to stimulate the sacral nerves, and thereby restore or control bodily functions affected by pelvic floor disorders. Several techniques of electrical neurostimulation may be used, including stimulation of nerve bundles within the sacrum. Such techniques may be particularly effective in alleviating sexual dysfunction or urinary incontinence. An example of an existing neurostimulation system for treatment of urinary urge incontinence is the implantable Interstim therapy system marketed by Medtronic, Inc. of Minneapolis, Minn.

Neurostimulation systems with multiple, self-contained neurostimulators also have been proposed. For example, U.S. Pat. No. 6,185,452 to Schulman et al. describes implantation of one or more miniature stimulators, referred to as microstimulators, with external electrodes for nerve or muscle stimulation. U.S. Pat. No. 6,650,943 to Whitehurst et al. describes implantation of microstimulators to treat erectile dysfunction. U.S. Pat. No. 6,735,474 to Loeb et al. describes a microstimulator system for treatment of urinary incontinence. Table 1 below lists documents that disclose various techniques for neurostimulation.

TABLE 1

| | Inventors/ | |
|---|---|---|
| Patent Number | Author | Title |
| 6,185,452 | Schulman | Battery-powered patient implantable |
| | et al. | device |
| 6,449,512 | Bojeva | Apparatus and method for treatment |
| | | of urological disorders using pro- |
| | | grammerless implantable pulse |
| | | generator system |
| 6,507,755 | Gozani et al. | Apparatus and method for stimu- |
| | | lating human tissue |
| 6,571,128 | Lebel et al. | Microprocessor controlled ambu- |
| | | latory medical apparatus with hand |
| | | held communication device |
| 6,650,943 | Whitehurst | Fully implantable neurostimulator |
| | et al. | for cavernous nerve stimulation as |
| | | a therapy for erectile dysfunction |
| | | and other sexual dysfunction |
| 6,735,474 | Loeb et al. | Implantable stimulator system and |
| | | method for treatment of inconti- |
| | | nence and pain |
| US20040019369 | Duncan et al. | Wireless functional electrical |
| | | stimulation system |

All documents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the techniques of the present invention.

### SUMMARY

The invention is directed to a neurostimulation system and method that make use of an array of distributed electrical stimulators implanted at selected positions within a patient. Each stimulator is capable of independently delivering neurostimulation energy to a different site within a patient. A master controller communicates with the stimulators by wireless telemetry, and synchronizes the operation of the stimulators to deliver a desired mode of neurostimulation energy to the patient. The distributed stimulators function as “slave” stimulators that selectively stimulate particular nerve sites at particular times. The master controller may activate different sets of stimulators during the source of a physiological activity. The stimulators or the master controller may be responsive to one or more physiological sensing devices also implanted within the patient.

Various embodiments of the present invention provide solutions to one or more problems existing in the prior art with respect to prior art systems for neurostimulation. These problems include difficulties associated with effectively treating different phases or components of a physiological activity, such as sexual activity or urinary activity. Sexual activity, for example, generally involves two distinct phases, arousal and orgasm. Urinary activity involves retention and voiding phases. In each case, the particular neurostimulation characteristics, such as site, timing, or pulse parameters, necessary to support and transition between such phases may be markedly different. Hence, a neurostimulation system may focus on stimulation to achieve one functional phase, but neglect others, resulting in reduced therapeutic efficacy for the patient receiving the neurostimulation. In addition, a neurostimulation system may be directed to one component that drives a particular physiological activity, but ignore other components, such as the contributions of multiple nerve sites.

Various embodiments of the present invention are capable of solving at least some of the foregoing problems. When embodied in a system or method for neurostimulation, the invention includes features that support the selective application of neurostimulation to target particular nerve sites at particular times, which may enable delivery of neurostimulation targeted to specific functional phases and components of a physiological activity. In accordance with the invention, a plurality of distributed stimulators operate on a coordinated basis to deliver different modes of neurostimulation energy to particular nerve sites at particular times. The timing and location of the neurostimulation energy delivered by the stimulators are selected to support distinct phases of physiological activity in a progressive manner, or to target a combination of different components, such as different nerve sites. In some embodiments, a master controller or the individual stimulators are responsive to signals generated by one or more sensing devices. The sensing devices sense physiological parameters that may be useful in identifying the state or phase of activity, or a transition between different phases of activity, and hence a triggering event for adjustment of the neurostimulation mode.

In one embodiment, the invention provides a method for delivering neurostimulation therapy. The method may comprise applying first neurostimulation therapy to a patient via a first set of one or more implanted stimulators, and applying second neurostimulation therapy to the patient via a second set of one or more implanted stimulators. At least some of the stimulators in the first set are positioned at sites that are different from sites at which at least some of the stimulators in the second set are positioned.

In another embodiment, the invention provides a neurostimulation system comprising a first set of one or more implantable stimulators for delivery of a first neurostimulation therapy to a patient, and a second set of one or more implanted stimulators for delivery of a second neurostimulation therapy to the patient via a second set of one or more implanted stimulators. At least some of the stimulators in the first set are positioned at sites that are different from sites at which at least some of the stimulators in the second set are positioned. A controller selectively activates the first set of stimulators to deliver the first neurostimulation therapy and the second set of stimulators to deliver the second neurostimulation therapy.

In comparison to known implementations for neurostimulation, various embodiments of the present invention may provide one or more of advantages. By addressing distinct phases or components of physiological activity, for example, the invention may provide a more effective neurostimulation therapy. Instead of applying a single neurostimulation therapy, or a neurostimulation therapy targeted to a single phase or component of physiological activity, the invention can provide more effective coverage during the course of the physiological activity. For example, the invention may produce a composite neurostimulation therapy that targets different nerve sites, simultaneously or at different times, with the aid of an array of distributed stimulators. In addition, the invention may provide a more orderly transition between distinct phases of physiological activity, and more effective restoration and support of the distinct phases. As a further advantage, using distributed, wireless stimulators, multi-site stimulation can be achieved without the need to implant an excessive number of leads within the patient.

The above summary of the present invention is not intended to describe each embodiment or every embodiment of the present invention or each and every feature of the invention. Advantages and attainments, together with a more complete understanding of the invention, will become apparent and appreciated by referring to the following detailed description and claims taken in conjunction with the accompanying drawings.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram illustrating an implantable neurostimulation system for delivery of neurostimulation to treat pelvic floor disorders.

FIG. 2 is a schematic diagram illustrating coordinated control of different sets of implanted stimulators.

FIG. 3 is a schematic diagram illustrating an implantable stimulator.

FIG. 4 is a schematic diagram illustrating an implantable sensing device.

FIG. 5 is a schematic diagram illustrating an implantable module incorporating a stimulator and a sensor.

FIG. 6 is a block diagram illustrating components of an implantable stimulator.

FIG. 7 is a block diagram illustrating components of an implantable sensing device.

FIG. 8 is a flow diagram illustrating a method for delivering neurostimulation therapies using first and second sets of stimulators in response to patient input.

FIG. 9 is a flow diagram illustrating a method for delivering neurostimulation therapies using first and second sets of stimulators in response to a sensed physiological signal.

FIG. 10 is a flow diagram illustrating a method for delivering neurostimulation therapies using first and second sets of stimulators in response to a timing event.

FIG. 11 is a flow diagram illustrating a method for delivering neurostimulation therapies using first and second sets of stimulators in greater detail.

### DETAILED DESCRIPTION OF THE PREFERRED EMBODIMENTS

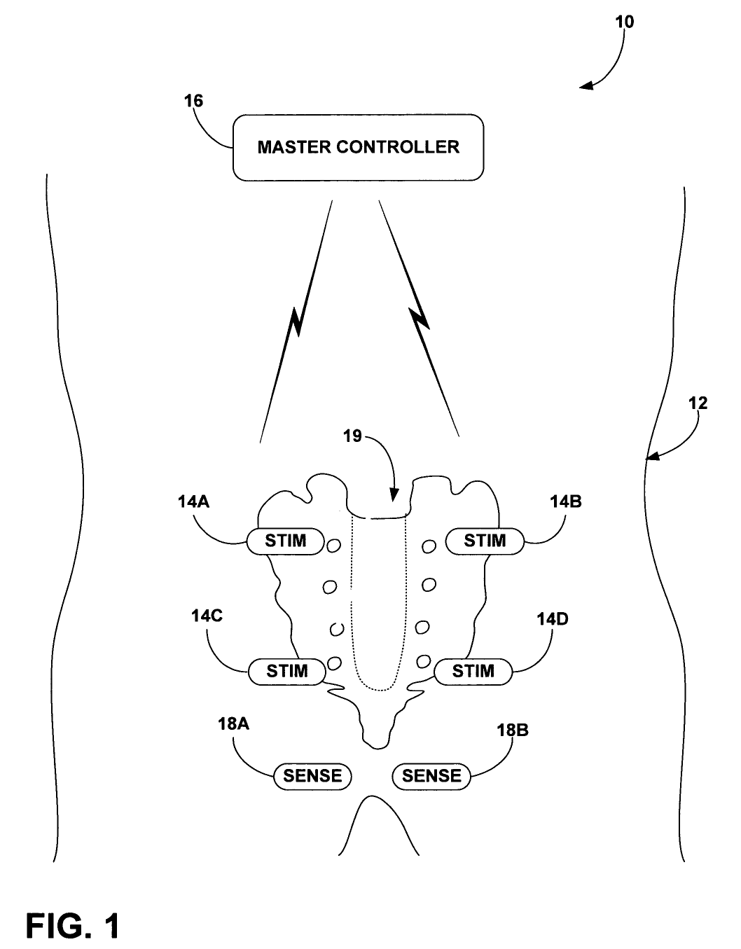

FIG. 1 is a schematic diagram illustrating an implantable neurostimulation system 10 for delivery of neurostimulation to treat pelvic floor disorders. System 10 is configured to deliver neurostimulation therapies to a patient 12 via a plurality of distributed stimulators 14A-14D (collectively stimulators 14) that may be capable of delivering neurostimulation energy independently of one another to different nerve sites. A master controller 16 communicates with stimulators 14 via wireless telemetry to control the operation of the stimulators in a selective, coordinated manner.

As further shown in FIG. 1, system 10 may include one or more distributed sensing devices 18 that sense physiological conditions within patient 12. Sensing devices 18 communicate information representing physiological conditions to master controller 16 by wireless telemetry. Sensing devices 18 may be implanted within the pelvic floor region or elsewhere to sense physiological conditions pertinent to the control of neurostimulation therapy delivered by stimulators 14.

Master controller 16 may be an external controller carried by patient 12. Alternatively, master controller 16 may be integrated within one of stimulators 14 or sensing devices 18. In this case, one of stimulators 14 acts as a “master” for one or more “slave” stimulators. Master controller 16 selectively activates and deactivates individual stimulators 14 or different sets of stimulators to deliver desired neurostimulation therapies to particular nerve sites at particular times. Master controller 16 may rely on information received from sensing devices 18 to selectively activate and deactivate stimulators 14.

The physiological conditions sensed by sensors 18 may be useful in identifying a state or phase of physiological activity, or a transition between different phases of activity. Master controller 16 may use information received from sensing devices 18 as a triggering event for adjustment of neurostimulation. Adjustment may include selective activation or deactivation of different sets of neurostimulators 14 or adjustment of neurostimulation parameters such as electrode polarity, voltage or current amplitude, frequency, pulse width and duration. Master controller 16 may rely on other triggering events, such as user input or timing information, either individually or in combination with other triggering events.

The selective activation of different sets of stimulators 14 enables delivery of neurostimulation energy targeted to specific functional phases and components of a physiological activity. For example, the timing and location of the neurostimulation energy delivered by the stimulators 14 can be selected to support distinct phases of physiological activity in a progressive manner, or to target a combination of different components, such as different nerve sites, that may contribute to the progress of a particular physiological activity. In one embodiment, master controller 16 controls a first set of stimulators 14 to apply a first neurostimulation therapy to patient 12, and controls a second set of stimulators to apply a second neurostimulation therapy to the patient. In this case, at least some of the stimulators 14 in the first set are positioned at sites that are different from sites at which at least some of the stimulators in the second set are positioned.

In the example of FIG. 1, stimulators 14 are implanted at different positions in proximity to sacrum 19 to access nerve sites within the pelvic floor region. In the example of FIG. 1, stimulators 14A and 14B are located proximate an upper region of sacrum 19, while stimulators 14C and 14D are implanted proximate a lower region of sacrum 19. Accordingly, stimulators 14A and 14B may stimulate a different set of nerves than stimulator 14C and 14D. As an example, stimulators 14A and 14B may target sacral nerves, while stimulators 14C and 14D target the pudendal nerve.

System 10 may be applied to deliver a variety of therapies formulated for different disorders or symptoms. Selected pelvic floor disorders such as sexual dysfunction or urinary incontinence will be described herein for purposes of illustration, although the invention is more broadly applicable to a variety of disorders that may respond to neurostimulation therapy. For example, distributed stimulators 14 within system 10 may cooperate to deliver therapy for alleviation of pelvic floor disorders such as urinary control disorders, fecal control disorders, interstitial cystitis, sexual dysfunction, and pelvic pain. Also, system 10 may be useful for spinal cord stimulation, providing sets of stimulators 14 that are oriented at different positions relative to the spinal cord.

As one example, system 10 may be applied to deliver therapy for relief of sexual dysfunction. The sexual dysfunction may take a variety of forms, including retrograde ejaculation, premature ejaculation, an ejaculation, and general inability to achieve arousal or orgasm. In an exemplary embodiment, system 10 delivers neurostimulation to the sacral nerves or other regions of the spinal cord known to have an effect on sexual function. Alternatively, or in addition, some stimulators 14 within system 10 may be configured to deliver neurostimulation to the pudendal nerve, the pelvic splanchnic nerve, or the cavernosa nerve in the penis.

A system for delivery of neurostimulation therapy for sexual dysfunction is disclosed in commonly assigned U.S. patent application Ser. No. 10/441,784, to Martin Gerber, filed May 19, 2003, entitled “TREATMENT OF SEXUAL DYSFUNCTION BY NEUROSTIMULATION,” the entire content of which is incorporated herein by reference. The system described in the above-referenced application may be adapted to use distributed stimulators 14 or other components of system 10, as described herein. For sexual dysfunction, for example, system 10 may be adapted for delivery of different modes of neurostimulation to support the progress of, and transition between, distinct phases of sexual activity, such as arousal and orgasm. Selective activation of different sets of stimulators 14 positioned at different nerve sites may more effectively target components that support the particular phases.

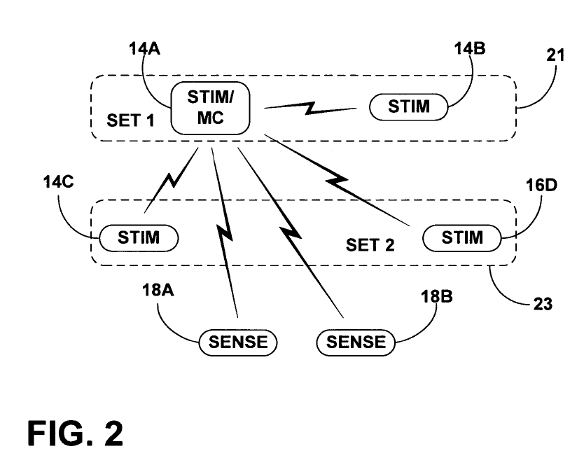

FIG. 2 is a schematic diagram illustrating coordinated control of different sets of implanted stimulators 14. In particular, FIG. 2 shows a first set 21 of stimulators 14, and a second set 23 of stimulators. In the example of FIG. 2, master controller 16 is integrated with stimulator 14A, although the master controller may be a separately implanted device or an external device carried by the patient 12. Integrated stimulator/master controller 14A acts as both a stimulator and a “master” controller for other “slave” stimulators 14. Stimulator 14A, as master controller, may be responsive to information received from implanted sensing devices 18 to generate control signals. Also, stimulator 14A may activate sensing devices 18 to obtain physiological information.

One or more stimulators 14 in system 10 are selectively implanted at positions designed to stimulate different C-fibers or sacral nerves at the second, third, and fourth sacral nerve positions, commonly referred to as S2, S3, and S4, respectively. Also, in some embodiments, one or more stimulators 14 may be implanted to deliver neurostimulation energy to the pudendal nerve. In this manner, system 10 may selectively stimulate the sacral nerves or pudendal nerve via different sets 21, 23 of stimulators 14.

For purposes of example, first set 21 of stimulators 14 is implanted proximate selected sacral nerves, while second set 23 is implanted proximate the pudendal nerve. In operation, stimulator/master controller 14A activates first set 21 of stimulators 14 with a first set of neurostimulation parameters to support the first phase of sexual activity. Then, stimulator/master controller 14A deactivates the first set 21 of stimulators 14, and activates second set 23 of stimulators with a second set of neurostimulation parameters to support the second phase of sexual activity.

Notably, in some embodiments, the first set 21 and second set 23 of stimulators 14 need not be mutually exclusive. For example, some stimulators 14 may be within both the first set and second set. Also, neurostimulation energy delivered by stimulators 14 in the first set may temporally or spatially overlap with neurostimulation energy delivered by stimulators in the second set. Moreover, the invention is not limited to delivery of stimulation via two sets of stimulators 14, but may encompass two or more sets of distributed stimulators.

Again, stimulator/master controller 14A activates first set 21 of stimulators 14 positioned proximate selected sacral nerves to initially deliver electrical stimulation with a first set of stimulation parameters selected to achieve a first phase of sexual activity. The first phase of sexual activity may involve sexual stimulation or arousal, which may be manifested by feelings of desire, penile erection in the case of male patients, or engorgement and lubrication in the case of female patients. The first set of stimulation parameters may specify the electrode polarity, waveform, voltage or current amplitude, pulse width, and frequency selected to support the first phase of sexual activity.

Upon receipt of a triggering event, which may include user input, timing information, or physiological information, stimulator/master controller 14A then activates second set 23 of stimulators 14 to support a second phase of sexual activity. For example, stimulator/master controller 14A activates stimulators 14 positioned proximate the pudendal nerve to cause the sexual activity to progress from the arousal phase, to a second phase, e.g., ejaculation or female orgasm. In addition to targeted stimulation of different nerve sites, the second set of distributed stimulators 14 may operate according to a second set of stimulation parameters appropriate to trigger the second phase of sexual activity.

Although the different sets of distributed stimulators 14 may target distinct phases of physiological activity, they also may work together to target different components that contribute to a single phase of activity. As an example, different sets of stimulators 14 may work together to simultaneously stimulate both the sacral nerves and the pudendal nerve to achieve a greater overall effect in restoring sexual function. In this sense, stimulation by second set 23 of stimulators 14 may be layered on top of stimulation provided by first set 21 of stimulators 14. In either case, the distributed stimulators are selectively activated and used in a coordinated manner to provide either a multi-function stimulation generator or a multi-site stimulator.

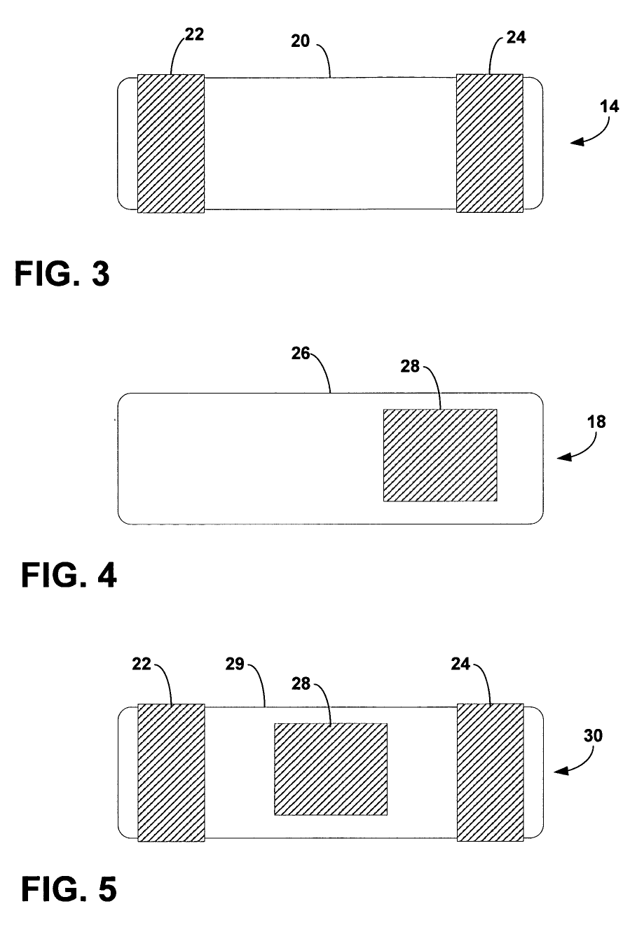

FIG. 3 is a schematic diagram illustrating an implantable stimulator 14. As shown in FIG. 3, stimulator 14 is preferably a self-contained module, mounted within its own housing 20. Housing 20 may be constructed from any of a variety of biocompatible materials, such as titanium. As will be described, housing 20 may carry one or more electrodes to permit delivery of electrical stimulation, an implantable pulse generator (IPG), and a telemetry interface to transmit or receive control signals or sensor signals. Although stimulator 14 may include short leads with electrodes that extend from the housing for placement proximate to a desired tissue or nerve site, the electrodes preferably are integrated with the stimulator.

In the example of FIG. 3, housing 20 carries a pair of electrodes 22, 24. Electrodes 22, 24 may be pads that are mounted on a particular surface of housing 20, or ring electrodes that extend about the entire periphery of the housing. Each stimulator 14 includes an implantable pulse generator, and delivers neurostimulation therapy to patient 12 via electrodes 22, 24 in the form of electrical pulses generated by the implantable pulse generator. In some cases, housing 20 itself may form an active “can” electrode.

In alternative embodiments, stimulator 14 may include a single electrode for coordinated operation with an external reference electrode, such as a ground pad. Alternatively, a stimulator 14 may include two or more electrodes that form a bipolar or multi-polar stimulation arrangement. Hence, stimulators 14 may deliver neurostimulation energy independently of other stimulators or in a coordinated manner with other stimulators. In either case, the electrode or electrodes may be formed on the housing 20 of stimulator 14.

FIG. 4 is a schematic diagram illustrating an implantable sensing device 18. Like stimulator 14, sensing device 18 preferably is a self-contained module having a housing 26. Like housing 20, housing 26 may be constructed from a biocompatible metal, such as titanium. A sensor 28 is mounted on or exposed by housing 26 to sense physiological conditions within patient 12 in the vicinity of the sensor. FIG. 5 is a schematic diagram illustrating an implantable module 30 incorporating both a stimulator and a sensor. In the example of FIG. 5, a housing 29 carries both electrodes 22, 24 and a sensor 28 to provide stimulation and sensing functionality within a single module. Integration of stimulation and sensing in a single module may be desirable in some applications. In other applications, however, it will be advantageous to sense physiological conditions at a location remote from the site of stimulation, in order to assess the response of patient 12 to the stimulation.

Distributed sensing device 18 permit the sensing of physiological conditions at different locations during the course of physiological activity. As an example, various types of sensing devices 18 may be useful in indicating the progress of sexual activity. In response to signals transmitted by a sensing device 18, a master controller 16 may selectively activate different sets 21, 23 of distributed stimulators 14. The different sets 21, 23 of stimulators 14 deliver different neurostimulation therapies in distinct phases of sexual activity with neurostimulation parameters selected as appropriate to support those phases.

As an example, one or more physiological parameters may be sensed by sensing devices 18 during a first phase, e.g., arousal, in which neurostimulation is delivered using the first set 21 of stimulators 14. The sensed physiological parameters are evaluated by sensing devices 18 or master controller 16 to determine that the patient is ready for progression to the next phase of sexual activity, e.g., orgasm. In this manner, master controller 16 obtains physiological parameters from different locations within the patient's body, such as within the genital area, via wireless telemetry by distributed sensing devices 18.

Suitable physiological parameters include, without limitation, pressure, electromyographic potentials, blood pressure, blood flow, penile size, tumescence, or the like. Alternatively, as will be described, patient 12 may provide an explicit indication that he or she is ready for progression to the second phase of sexual activity. As a further alternative, a timer may be employed to indicate progression to the second phase of sexual activity following expiration of a predetermined period of time after initiation of the first phase of sexual activity.

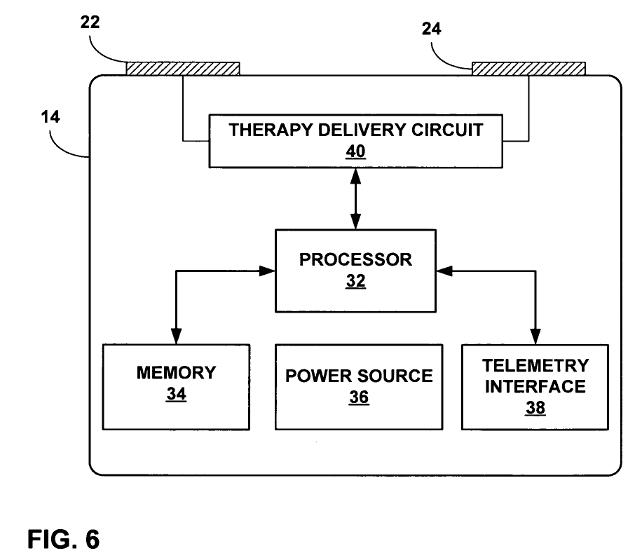

FIG. 6 is a block diagram illustrating various components of an implantable stimulator 14 for use on a distributed basis within system 10 of FIG. 1. In the example of FIG. 6, a stimulator 14 includes a housing carrying a pair of electrodes 22, 24, which can be referenced to each other to form a bipolar arrangement. Stimulator 14 further includes a processor, memory 34, power source 36, telemetry interface 38, and therapy delivery circuit 40. Electrodes 22, 24 are electrically coupled to a therapy delivery circuit 40, which includes an implantable pulse generator to generate stimulation pulses. Power source 36 may be a battery, which may be non-rechargeable. Alternatively, the battery may be rechargeable with power delivered from an external charging device via an inductive power interface. As a further alternative, stimulator 14 may be inductively powered by an external device.

Processor 32 controls the implantable pulse generator within therapy delivery circuit 40 to deliver neurostimulation therapy according to selected stimulation parameters. Specifically, processor 32 controls therapy delivery circuit 40 to deliver electrical pulses with selected voltage or current amplitudes, pulse widths, frequencies, and durations specified by programs stored in memory 34. In addition, processor 32 may control therapy delivery circuit 40 to deliver neurostimulation pulses via one or both of electrodes 22, 24 with selected polarities. In some embodiments, two or more electrodes may be provided on the housing of stimulator 14.

Processor 32 may control therapy delivery circuit 40 to deliver each pulse according to a different program, thereby interleaving programs to simultaneously treat different symptoms or provide a combined therapeutic effect. For example, in addition to treatment of sexual dysfunction, stimulator 14 may be configured to deliver neurostimulation therapy to simultaneously treat pain or incontinence. Processor 32 may include a microprocessor, a controller, a digital signal processor (DSP), an application specific integrated circuit (ASIC), a field programmable gate array (FPGA), or other equivalent logic circuitry, or the like.

In some embodiments, memory 34 stores multiple sets of stimulation parameters that are available to be selected by patient 12 for delivery of neurostimulation therapy. For example, memory 34 may store stimulation parameters transmitted by an external clinician programmer. As described herein, the stimulation parameters may be formulated for treatment during distinct phases of sexual activity, such as a first phase involving arousal and a second phase involving orgasm. An external programmer may communicate with stimulator 14 by wireless telemetry to adjust neurostimulation delivered by the stimulator.

Memory 34 also stores program instructions that, when executed by processor 32, cause stimulator 14 to deliver neurostimulation therapy. Memory 34 may include any volatile or non-volatile media, such as a RAM, ROM, NVRAM, EEPROM, flash memory, and the like, or any combination thereof. Accordingly, the invention also contemplates computer-readable media storing instructions to cause processor 32 to provide the functionality described herein.

Telemetry interface 38 supports wireless communication between stimulator 14 and master controller 16 for coordinated control with other stimulators, as well as communication with an external clinician programmer or patient programmer for programming of the stimulator. A handheld computing device (not shown) may be provided as a programmer to permit a clinician to program neurostimulation therapy into stimulators 14 for patient 12, e.g., using input keys and a display. Using the external programmer, the clinician may specify neurostimulation parameters for use in the different phases of physiological activity by patient 12. The external programmer supports radio frequency telemetry with stimulators 14 to download neurostimulation parameters and, optionally, upload operational or physiological data from the stimulators and sensing devices 18. In this manner, a clinician may periodically interrogate system 10 to evaluate efficacy and, if necessary, modify the stimulation parameters.

The clinician programmer, in some embodiments, may be integrated with master controller 16. More preferably, master controller 16 is integrated with an external patient programmer or one of stimulators 14. Like the clinician programmer, a patient programmer can be provided as a handheld computing device. The patient programmer may include a display and input keys to allow patient 12 to interact with the patient programmer. In this manner, the patient programmer provides patient 12 with an interface for control of neurostimulation therapy by distributed stimulators 14. For example, patient 12 may use the patient programmer to start, stop or adjust neurostimulation therapy. In particular, the patient programmer may permit patient 12 to adjust stimulation parameters such as amplitude, frequency, pulse width and duration, within an adjustment range specified by the clinician via a clinician programmer.

In some embodiments, the patient programmer may permit patient 12 to explicitly control transition of neurostimulation therapy from a first phase of activity to a second phase of activity. The patient programmer, whether integrated with master controller 16, or not, supports radio frequency telemetry with stimulators 14 and sensing devices 18 to transmit neurostimulation instructions and receive sensed physiological conditions, and is sized for ease of portability, permitting patient 12 to carry the patient programmer.

Telemetry interface 38 may support wireless communication with one or more wireless sensing devices 18 that sense physiological signals and transmit the signals to stimulator 14. Hence, stimulator 14 may be directly responsive to physiological signals generated by sensing devices 18. Alternatively, master controller 16 may receive the physiological signals and transmit control signals to stimulator 14. As described above, master controller 16 may be responsive to physiological signals sensed by physiological sensing devices 18 to control delivery of neurostimulation by one or more stimulators 14. In response to detection of a particular physiological condition or level, master controller 16 may adjust the neurostimulation therapy delivered by stimulators 14.

For example, master controller 16 may transition from a first set of stimulators 14 used to support a first phase of sexual activity to a different set of stimulators 14 used to support a second phase of sexual activity. Alternatively, master controller 16 may adjust the stimulation parameters associated with neurostimulation therapy delivered by a given set of stimulators 14 during a respective phase of sexual activity. In a first phase of sexual activity, sensing devices 18 may transmit signals indicative of the response of patient 12 to existing neurostimulation parameters. As an illustration, an implanted or external sensing device 18 may indicate a reduction in penile tumescence, in which case master controller 16 may increase the amplitude or frequency of neurostimulation pulses delivered by the first set of stimulators 14 in order to increase tumescence and maintain the first phase of sexual activity. In this manner, sensing devices 18 provide closed loop feedback for control of neurostimulation therapy.

Sensing device 18 also may sense one or more physiological parameters indicative of progression from the first phase of sexual activity to the second phase of sexual activity. For example, physiological sensing device 18 may sense changes in pressure, electromyographic potentials, or tumescence as an indication that patient 12 is ready for the second phase. In response, master controller 16 activates the second set of stimulators 14 to support transition to the second phase. Master controller 16 also may deactivate the first set of stimulators 14, either before activation of the second set or after a predetermined period of time following activation of the second set. In some cases, first and second sets of stimulators 14 may be activated simultaneously, providing overlapping coverage. For example, the second set of stimulators 14 may be activated to provide an added layer of neurostimulation that triggers the second phase.

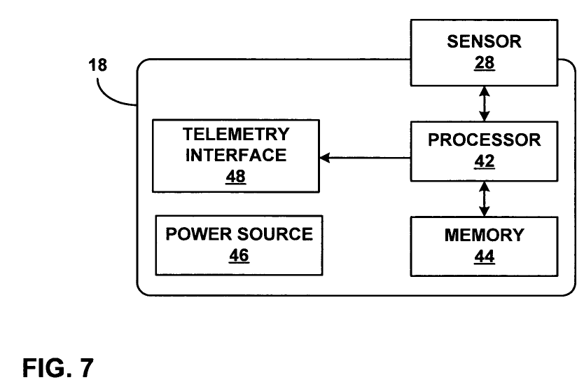

FIG. 7 is a block diagram illustrating various components of an implantable sensing device 18. As shown in FIG. 7, sensing device 18 may include a processor 42, memory 44, power source 46, telemetry interface 48 and physiological sensor 28. Processor 42 and memory 44 may not be necessary in some embodiments. Instead, sensing device 18 may simply provide a sensor 28 and telemetry interface 48 equipped to transmit a raw, unprocessed sensor signal to master controller 16 and/or distributed stimulators 14. In general, processor 42, memory 44, telemetry interface 48 and power source 46 may be constructed like similar components within stimulator 14, as described above with reference to FIG. 6.

For treatment of sexual dysfunction, sensing device 18 can be implanted within the genital region of patient 12, preferably without the need for a lead. For example, sensor 28 may be selected to sense pressure, blood flow, blood pressure, penile tumescence or the like. As further options, physiological sensor 18 may sense temperature, pressure changes, and frequency of pressure changes. Some sensing devices 18 may be deployed near the sacrum 19 to sense nerve responses.

Sensing devices 18 may be implanted within patient 12 or, in some cases, mounted externally. For example, in some embodiments, a penile tumescence, flow or pressure sensor may take the form of an external strain gauge ring mounted about the shaft of the penis. In various embodiments, sensing device 18 may take a variety of forms sufficient to sense desired physiological conditions including pressure sensors, flow sensors, temperature sensors, electromyographic sensors. Hence, in terms of structure, sensing device 18 may include strain gauge sensors, optical sensors, ultrasonic sensors, piezoelectric sensors, electrical sensors, or the like.

As one example, sensor 28 may take the form of a pressure sensor implanted within the penis or vagina of patient 12. The pressure sensor monitors pressure levels and transmits a wireless signal indicative of the pressure levels to stimulators 14 or master controller 16 via wireless telemetry. The pressure sensor may monitor, for example, urethral pressure or blood pressure. Master controller 16 processes the pressure level signal and determines whether the pressure level exceeds or falls below an applicable predetermined threshold. Alternatively, master controller 16 analyzes changes in the pressure signal, and compares the rate of change of frequency of change to applicable thresholds.

Monitoring changes in pressure may permit system 10 to obtain a parameter indicative of a rhythm associated with sexual activity. As the pressure level, pressure slope or frequency of changes in pressure level exceeds an applicable threshold, master controller 16 transitions from a first set of stimulators 14 for a first sexual phase to a second set of stimulators 14 for a second sexual phase.

As another example, sensor 28 may take the form of an electromyographic (EMG) sensor that measures EMG potentials within the genital region, e.g., within the penis or clitoris. Physiological sensing device 18, or sense electrodes associated with sensor 28, may be implanted within the genital region. In this case, master controller 16, or distributed sensing devices 18, receives EMG signals from physiological sensing device 18 via wireless communication, and processes the signals to identify EMG levels, slopes, or frequency of EMG changes that exceed applicable thresholds.

Physiological sensing device 18, according to another example, may take the form of a blood flow sensor that monitors increased blood flow into the male or female genital region, i.e., tumescence. In this case, physiological sensing device 18 can be implanted in the penis or vagina, and may sense tumescence by sensing impedance changes or pressure changes. For pressure changes, for example, a strain gauge may be fitted to the genital region, either over or under the skin. Impedance measurements on the skin surface also may be indicative of lubrication. Master controller 16 receives the tumescence signals from physiological sensing device 18, via a lead or wireless communication, and processes the signals to identify tumescence levels, slopes, or frequency of change that exceed applicable thresholds. When the threshold is exceeded, system 10 transitions between first and second sets of stimulators 14.

For urinary incontinence applications, sensing devices 18 may be implanted to sense bladder pressure, bladder contractile force, urine level, urethral pressure, urethral flow, urine presence within the urethra or other parameters indicating the state of bladder function. In a first phase of urinary activity, master controller 16 may activate a first set of stimulators 14 to cause bladder contraction and urinary voiding, and then activate a second set of stimulators 14 to cause bladder relaxation for retention of urine. Alternatively, a first set of stimulators 14 may cause urinary sphincter contraction to retain urine, while a second set of stimulators 14 causes sphincter relaxation to permit urine flow. In either case, sensing devices 18 may provide feedback to master controller 16 for maintenance of, or transition between, such phases of urinary activity.

Master controller 16, stimulators 14 and sensing devices 18 communicate via radio frequency (RF) telemetry. For example, RF telemetry may be accomplished using any of a variety of RF communication techniques and protocols, such as proprietary RF communication protocols used in the medical device arts, as well as standardized RF communication protocols in more general use, such as the various IEEE 802.11 protocols or the Bluetooth protocol. Master controller 16, stimulators 14 and sensing devices 18 are equipped with appropriate modulation, demodulation, amplification, filtering and antenna circuitry to support wireless telemetry.

In general, to facilitate collision-free, two-way communication, master controller 16 may assign time slots, frequency channels, or spreading codes to sensing devices 18. In this manner, sensing devices 18 can transmit sensor signals to master controller 16 without contention. Master controller 16 may statically assign time slots or channels to sensing devices 18 or change the assignments dynamically. For example, some sensing devices 18 may be less important during one phase of physiological activity, and therefore may be afforded less bandwidth for transmissions, whereas other sensing devices may receive greater bandwidth. Dynamic assignment of bandwidth may also permit conservation of battery resources during periods in which frequent transmission of sensor signals would be wasteful.

To activate different sets 21, 23 of stimulators 14 or sensing device 18, master controller 16 may employ an addressing scheme. For example, each stimulator 14 or sensors 18 may be assigned a unique address so that signals transmitted by the master controller 16 and intended for a particular stimulator or sensing device can be identified by the respective device. In other words, a particular stimulator 14 or sensing device 18 is responsive to control signals carrying the appropriate address or identifier.

In addition, to facilitate activation of a set of multiple stimulators 14 or sensing devices 18, master controller 16 may employ a group addressing scheme. For example, a set 21, 23 of stimulators 14 may be responsive to a group address identifier, which signifies that each stimulator sharing the group identifier should respond to the control signal. A similar approach may be used for sensing devices 18, if multiple sensing devices are to be activated in groups. In each case, a unique address or group address may be transmitted with a control signal, e.g., within a packet header or other administrative section of a transmission.

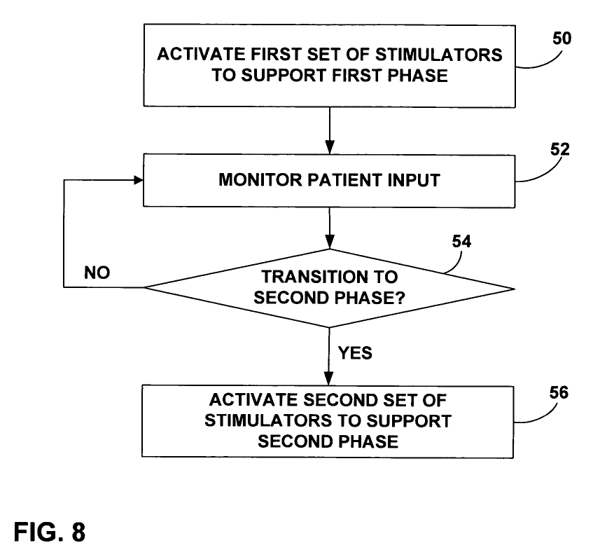

FIG. 8 is a flow diagram illustrating a method for delivering neurostimulation therapies using first and second sets of stimulators 14 in response to patient input. In the example of FIG. 8, master controller 16 activates a first set 21 of stimulators 14 to support a first phase of physiological activity (50), such as a first phase of sexual activity. The first phase may be initiated in response to user input requesting that master controller 16 activate neruostimulation. Master controller 16, which may be embodied within an external programmer or integrated with a stimulator 14 or sensing device 18, monitors user input (52). If the user input indicates a desire to transition form the first phase of activity to a second phase of physiological activity (54), master controller 16 activates a second set of stimulators 14 to support the second phase of physiological activity (56).

In this manner, system 10 is responsive to an explicit indication by patient 12 that he or she is ready for progression to the second phase of activity. The user input may be provided by actuating an input device associated with an external programmer, which may or may not incorporate master controller 16. For transition from the first phase to the second phase, master controller 16 also may selectively apply different stimulation parameters via distributed stimulators 14. The first set of stimulators 14 may remain activated with the second set of stimulators, with the same or different stimulation parameters. Alternatively, the first set of stimulators 14 may be deactivated upon or shortly following activation of the second set of stimulators.

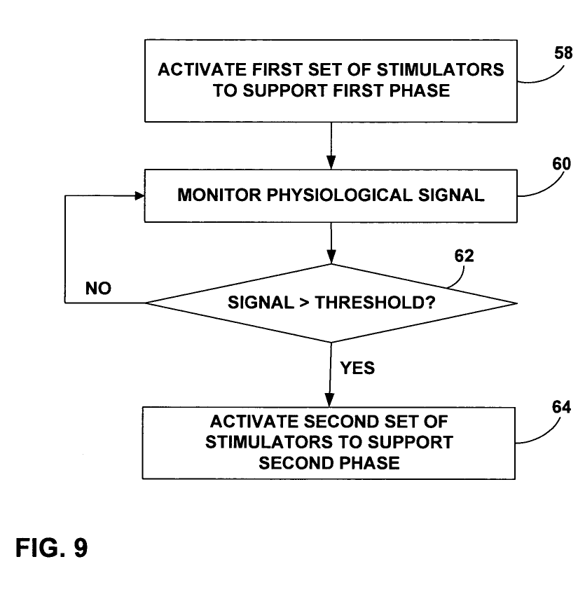

FIG. 9 is a flow diagram illustrating a method for delivering neurostimulation therapies using first and second sets of stimulators in response to a sensed physiological signal. The method of FIG. 9 involves monitoring a physiological signal during the course of physiological activity via one or more distributed sensing devices 18 and delivering stimulation via one or more distributed stimulators 14 in response to the sensed signal. As shown in FIG. 9, master controller 16 a first set 21 of stimulators 14 to support a first phase of physiological activity (58), and monitors a physiological signal transmitted by one or more of sensing devices 18 (60). If the sensed signal exceeds an applicable threshold (62), or otherwise satisfies a predetermined set of criteria, master controller 16 activates a second set of stimulators 14 to support a second phase of activity (64).

Master controller 16 may be responsive to a level of the sensed signal, or some other characteristic of the signal, such as a frequency, average or trend. In the example of sexual dysfunction, the signal may represent a variety of parameters such as pressure, blood flow, blood pressure, penile tumescence or the like. Master controller 16 may receive a raw signal and process the signal for comparison to a threshold or other criteria. Alternatively, sensing devices 18 may pre-process the signal for local comparison to a threshold or other criteria, and transmit a trigger signal to master controller 16.

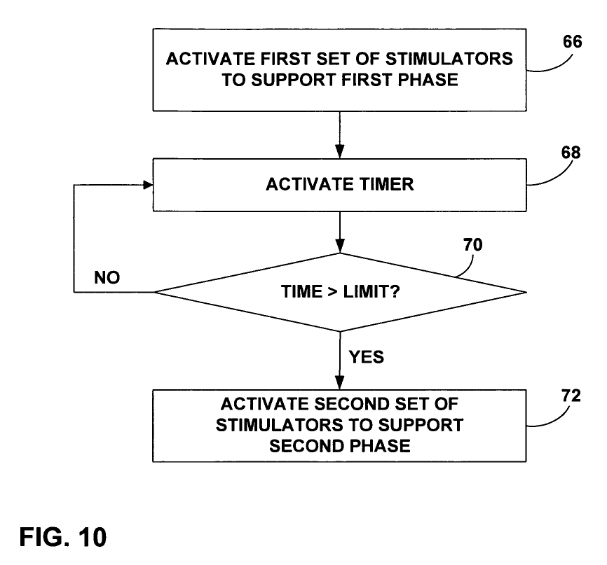

FIG. 10 is a flow diagram illustrating a method for delivering neurostimulation therapies using first and second sets of stimulators in response to a timing event. In the example of FIG. 10, master controller 16 activates a first set 21 of stimulators 14 to support a first phase of physical activity 66), and then activates a timer (68). When the timer reaches a predetermined limit (70), master controller 16 activates a second set 23 of stimulators 14 to support a second phase of activity (72). In this manner, master controller 16 applies a time limit between the first phase and the transition to the second phase. The timing information may be combined with other trigger events. For example, master controller 16 may consider both the time limit and physiological signals sensed by sensing device 18. Also, user input may override control based on a time limit or physiological signals to permit the user to transition between the phases at an earlier or later time.

In the case of sexual activity, master controller 16 applies a time limit between arousal and orgasm. For urinary activity, master controller 16 may apply a time limit between the start of a voiding event and the end of a voiding event. For example, first set 21 of stimulators 14 may initially trigger relaxation of the bladder sphincter and/or contraction of the bladder muscle, and the second set 23 may trigger contraction of the bladder sphincter and/or relaxation of the bladder muscle upon expiration of a time limit.

For urinary incontinence, neurostimulation for different phases of a voiding event may be accompanied by a substantially full-time neurostimulation program that counteracts bladder contraction or sphincter relaxation to avoid incontinence outside of planning voiding events. When the patient wishes to void, he may provide user input requesting that master controller 16 activate neurostimulation to permit voiding.

The time limit for sexual activity or voiding may be selected to correspond to an average time ordinarily associated with normal transition between the distinct phases of activity, or a time unique to a particular patient 12. For example, the predetermined period of time may be selected by a clinician according to a patient request. Alternatively, in some embodiments, patient 12 may be permitted to adjust the time using a patient programmer.

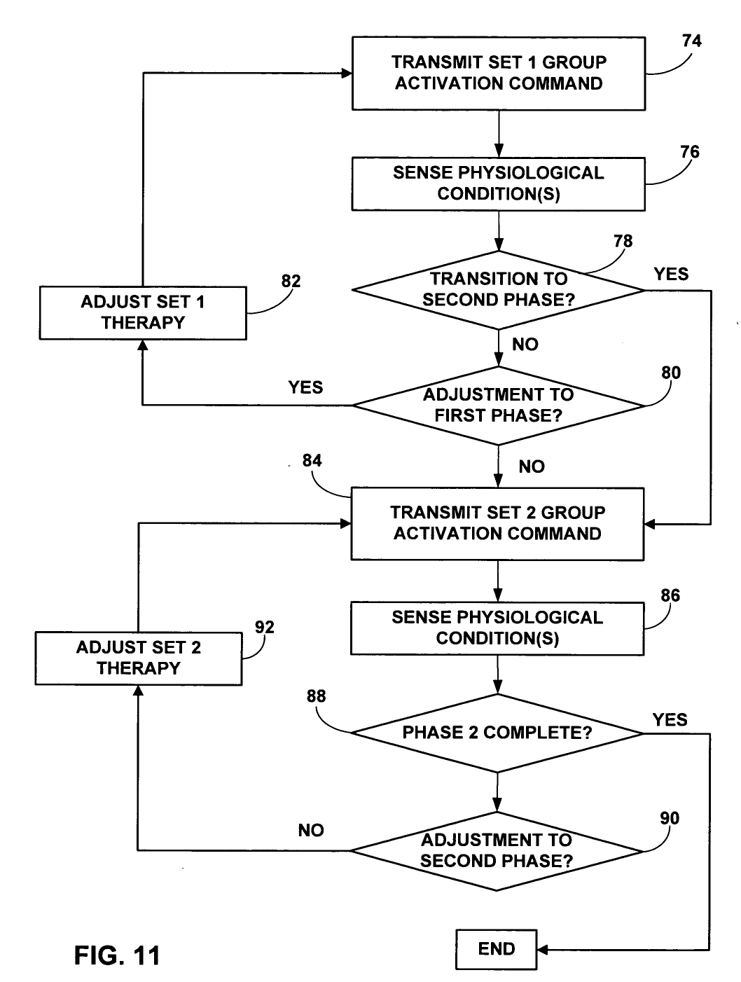

FIG. 11 is a flow diagram illustrating a method for delivering neurostimulation therapies using first and second sets of stimulators in greater detail. In the example of FIG. 11, master controller 16 controls not only selection of different set of stimulators 14 to transition between different phases, but also adjustment of neurostimulation parameters within a given phase. In addition, FIG. 11 depicts the transmission of group activation commands to activate a set of stimulators 14 simultaneously.

As shown in FIG. 11, master controller 16 initially transmits a group activation command to activate a first set of stimulators 14 (74) to deliver neurostimulation energy. During the course of the first phase, sensing devices 18 sense physiological conditions (76) and transmit corresponding signals to master controller 16. If the signals do not indicate the need for transition to a second phase (78), master controller 16 determines whether the signals indicate the need for an adjustment to the neurostimulation parameters within the first phase (80). If so, master controller 16 adjusts one or more neurostimulation parameters (82) and transmits the adjustments to the first set 21 of stimulators 14 in a group activation command (74).

The sensing of physiological conditions and adjustment of neurostimulation parameters continues on an iterative, closed loop basis until a transition to the second phase is necessary (78), e.g., in response to a sensed physiological condition or in response to another trigger event such as expiration of a time limit or an explicit user command. For transition to the second phase, master controller 16 transmits a group activation command (84) to activate a second set 23 of stimulators 14. Master controller 16 then initiates a closed loop adjustment process for adjustment of the neurostimulation parameters delivered by the second set of stimulators 14, until the physiological activity is complete.

For example, during the course of the second phase, sensing devices 18 sense physiological conditions (86) and transmit corresponding signals to master controller 16. If the signals do not indicate completion of the second phase (88), master controller 16 determines whether the signals indicate the need for an adjustment to the neurostimulation parameters within the second phase (90), e.g., to achieve completion. If so, master controller 16 adjusts one or more neurostimulation parameters (92) and transmits the adjustments to the second set 23 of stimulators 14 in another group activation command (84).

As an illustration, for sexual dysfunction, master controller 16 controls a first set 21 of stimulators 14 to deliver neurostimulation pulses to selected sacral nerves to support a first phase of sexual activity, i.e., arousal. The neurostimulation pulses delivered by the first set 21 of stimulators 14 may have a frequency in the range of approximately 10 to 150 Hz, and more preferably approximately 20 to 60 Hz. Each pulse for the first phase may have an voltage amplitude in the range of approximately 0.5 to 10 volts, and more preferably approximately 2 to 5 volts, and a pulse width in the range of approximately 100 to 400 microseconds, and more preferably approximately 200 to 300 microseconds. The duration of the first phase neurostimulation will depend on the detected transition to the second phase, but typically may be on the order of approximately 2 to 30 minutes. Master controller 16 or stimulators 14 may specify multiple settings, however, such that there are different sets of parameters for the first phase for different times of the day, different environments, and the like.

Upon transition to the second phase, master controller 16 controls a second set of stimulators 14 to deliver neurostimulation pulses to a different location within the pelvic floor region. In particular, at least some of the stimulators 14 in the second set 23 are positioned at sites that are different from sites at which at least some of the stimulators 14 in the first set 21 are positioned. For example, one or more of stimulators 14 in the second set 23 may be placed near the pudendal nerve. The pulses delivered by simulators 14 in the second set 23 may have a frequency in the range of approximately 1 to 5 Hz, or in the range of approximately 25 to 35 Hz. Each pulse for the second phase may have an amplitude in the range of approximately 1 to 10 volts, and more preferably approximately 2 to 5 volts, and a pulse width in the range of approximately 100 to 700 microseconds, and more preferably approximately 200 to 300 microseconds. The duration of the second phase neurostimulation delivered by the second set 23 of stimulators 14 may be on the order of approximately 1 to 5 minutes. Like the first phase neurostimulation parameters, there may be multiple settings for the second phase parameters.

The preceding specific embodiments are illustrative of the practice of the invention. It is to be understood, therefore, that other expedients known to those skilled in the art or disclosed herein may be employed without departing from the invention or the scope of the claims. For example, the present invention is not limited to the particular factors of sexual dysfunction described herein. In addition, the present invention further includes within its scope methods of making and using systems for neurostimulation, as described herein. Importantly, although application of the invention to sexual dysfunction and urinary incontinence has been described herein, the invention may be broadly applicable to a variety of other disorders such as chronic pain, urinary or fecal incontinence, interstitial cystitis, and pelvic pain, to name a few. When applied to nerves found to stimulate sexual activity, the invention may be applied to sacral nerves, the pudendal nerve, the pelvic splanchnic nerve, or the cavernosa nerve in the penis. In addition, although wireless communication is contemplated between distributed stimulators 14, master controller 16, and sensing devices 18, in some embodiments, communication may be accomplished, at least in part, by wired connections.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A method for delivering neurostimulation therapy, the method comprising:
applying first neurostimulation therapy to a patient via a first set of one or more implanted stimulators; and
applying second neurostimulation therapy to the patient via a second set of one or more implanted stimulators,
wherein at least some of the stimulators in the first set are positioned at sites that are different from sites at which at least some of the stimulators in the second set are positioned.

2. The method of claim 1, further comprising applying the first and second neurostimulation therapies at different times.

3. The method of claim 1, further comprising applying the first neurostimulation therapy to support a first phase of physiological activity by the patient, and applying the second neurostimulation therapy to support a second phase of physiological activity.

4. The method of claim 1, wherein the first and second phases of physiological activity are first and second phases of sexual activity.

5. The method of claim 1, wherein the first phase includes sexual arousal and the second phase includes sexual orgasm.

6. The method of claim 1, wherein the first and second phases of physiological activity are first and second phases of urinary activity.

7. The method of claim 1, wherein the first phase includes urinary retention and the second phase includes urinary voiding.

8. The method of claim 1, further comprising sensing one or more physiological conditions within the patient, and adjusting at least one of the first neurostimulation therapy and the second neurostimulation therapy based on the sensed physiological conditions.

9. The method of claim 8, wherein sensing includes sensing the physiological conditions with an implanted sensor and transmitting a sensor signal representing the sensed physiological conditions by wireless communication to at least one of the stimulators.

10. The method of claim 9, further comprising adjusting at least one of the first and second neurostimulation therapies in response to the transmitted sensor signal.

11. The method of claim 9, further comprising deactivating application of the first neurostimulation therapy and activating application of the second neurostimulation therapy in response to the transmitted sensor signal.

12. The method of claim 8, further comprising sensing the one or more physiological conditions via one of a plurality of implanted sensors.

13. The method of claim 8, wherein the one or more sensed physiological conditions include at least one of penile pressure, penile electromyographic potential, penile blood flow, penile tumescence, and penile size.

14. The method of claim 8, wherein the one or more sensed physiological conditions include at least one of bladder flow, bladder electromyographic potential, bladder pressure, and urethral flow.

15. The method of claim 1, further comprising deactivating the first neurostimulation energy and activating the second neurostimulation for transition from a first phase of physiological activity to a second phase of physiological activity.

16. The method of claim 1, further comprising deactivating the first neurostimulation therapy and activating the second neurostimulation in response to a triggering event.

17. The method of claim 16, wherein the triggering event includes a user command, a change in a physiological parameter, or a timing event.

18. The method of claim 17, wherein the triggering event includes a combination of at least two of a user command, a change in a physiological parameter, or a timing event.

19. The method of claim 1, wherein the first neurostimulation therapy includes a set of neurostimulation parameters that are different from a set of neurostimulation parameters associated with the second neurostimulation therapy.

20. The method of claim 19, wherein the neurostimulation parameters include amplitude, pulse width, frequency, and duration.

21. The method of claim 1, wherein the stimulators are positioned for application of the first and second neurostimulation therapies to sacral nerves of the patient.

22. The method of claim 1, wherein the stimulators are positioned for application of the first and second neurostimulation therapies to at least one of the S2 and S3 sacral nerves of the patient.

23. The method of claim 1, wherein the stimulators are positioned for application of at least one of the first and second neurostimulation therapies to a pudendal nerve of the patient.

24. The method of claim 1, wherein the stimulators are positioned for application of one of the first and second neurostimulation therapies to the sacral nerves and the other of the first and second neurostimulation therapies to the pudendal nerve.

25. The method of claim 1, wherein the stimulators are positioned for application of the first neurostimulation therapy to the sacral nerves to support sexual arousal, and the second neurostimulation therapy to the pudendal nerve to support orgasm.

26. The method of claim 1, wherein the stimulators are positioned for application of the first neurostimulation therapy to the sacral nerves, and the second neurostimulation therapy to the pudendal nerve.

27. The method of claim 1, further comprising controlling the stimulators with one of the stimulators.

28. The method of claim 1, further comprising controlling the stimulators with an external controller.

29. The method of claim 1, wherein each of the stimulators includes a self-contained, self-powered stimulator module with at least two external electrodes.

30. The method of claim 29, wherein some of the stimulators further include a sensor to sense one or more physiological conditions within the patient.

31. A neurostimulation system comprising:
a first set of one or more implantable stimulators for delivery of a first neurostimulation therapy to a patient;
a second set of one or more implanted stimulators for delivery of a second neurostimulation therapy to the patient via a second set of one or more implanted stimulators; and
a controller to selectively activate the first set of stimulators to deliver the first neurostimulation therapy and the second set of stimulators to deliver the second neurostimulation therapy,
wherein at least some of the stimulators in the first set are positioned at sites that are different from sites at which at least some of the stimulators in the second set are positioned.

32. The system of claim 31, wherein the controller selectively activates the first and second sets of stimulators at different times.

33. The system of claim 31, wherein the controller activates the first set of stimulators to apply the first neurostimulation therapy to support a first phase of physiological activity by the patient, and activates the second set of stimulators to apply the second neurostimulation therapy to support a second phase of physiological activity.

34. The system of claim 31, wherein the first and second phases of physiological activity are first and second phases of sexual activity.

35. The system of claim 31, wherein the first phase includes sexual arousal and the second phase includes sexual orgasm.

36. The system of claim 31, wherein the first and second phases of physiological activity are first and second phases of urinary activity.

37. The system of claim 31, wherein the first phase includes urinary retention and the second phase includes urinary voiding.

38. The system of claim 31, further comprising one or more implantable sensors to sense one or more physiological conditions within the patient, wherein the controller adjusts at least one of the first neurostimulation therapy and the second neurostimulation therapy based on the sensed physiological conditions.

39. The system of claim 38, wherein each of the sensors includes a wireless transmitter to transmit a sensor signal representing the sensed physiological conditions to the controller.

40. The system of claim 39, wherein the controller adjusts at least one of the first and second neurostimulation therapies in response to the transmitted sensor signal.

41. The system of claim 39, wherein the controller deactivates the first set of stimulators to cease application of the first neurostimulation therapy and activates the second set of stimulators to apply the second neurostimulation therapy in response to the transmitted sensor signal.

42. The system of claim 38, wherein the one or more sensed physiological conditions include at least one of penile pressure, penile electromyographic potential, penile blood flow, penile tumescence, and penile size.

43. The system of claim 38, wherein the one or more sensed physiological conditions include at least one of bladder flow, bladder electromyographic potential, bladder pressure, and urethral flow.

44. The system of claim 31, wherein the controller deactivates the first set of stimulators to cease application of the first neurostimulation therapy and activates the second set of stimulators to apply the second neurostimulation therapy for transition from a first phase of physiological activity to a second phase of physiological activity.

45. The system of claim 31, wherein the controller deactivates the first set of stimulators to cease application of the first neurostimulation therapy and activates the second set of stimulators to apply the second neurostimulation therapy in response to a triggering event.

46. The system of claim 45, wherein the triggering event includes a user command, a change in a physiological parameter, or a timing event.

47. The system of claim 46, wherein the triggering event includes a combination of at least two of a user command, a change in a physiological parameter, or a timing event.

48. The system of claim 31, wherein the first neurostimulation therapy includes a set of neurostimulation parameters that are different from a set of neurostimulation parameters associated with the second neurostimulation therapy.

49. The system of claim 48, wherein the neurostimulation parameters includes amplitude, pulse width, frequency, and duration.

50. The system of claim 31, wherein the stimulators are positioned for application of the first and second neurostimulation therapies to sacral nerves of the patient.

51. The system of claim 31, wherein the stimulators are positioned for application of the first and second neurostimulation therapies to at least one of the S2 and S3 sacral nerves of the patient.

52. The system of claim 31, wherein the stimulators are positioned for application of at least one of the first and second neurostimulation therapies to a pudendal nerve of the patient.

53. The system of claim 31, wherein the stimulators are positioned for application of one of the first and second neurostimulation therapies to the sacral nerves and the other of the first and second neurostimulation therapies to the pudendal nerve.

54. The system of claim 31, wherein the stimulators are positioned for application of the first neurostimulation therapy to the sacral nerves, and the second neurostimulation therapy to the pudendal nerve.

55. The system of claim 31, wherein the controller resides within one of the stimulators, the respective stimulator include a wireless transmitter to transmit commands to the other stimulators.

56. The system of claim 31, wherein the controller includes an external controller carried by the patient.
